(19) United States
(12) Patent Application Publication
(10) Pub. No.: US 2008/0293728 A1
(43) Pub. Date: Nov. 27, 2008

(54) Complexes Comprising alpha2-Adrenergic Receptor Agonists and Compositions

(76) Inventors: Gregory L. McIntire, Rochester, NY (US); Stephen R. Davio, Fairport, NY (US); Arthur E. Harms, Overland Park, KS (US); Hongna Wang, Fairport, NY (US)

Correspondence Address: Bausch & Lomb Incorporated, One Bausch & Lomb Place, Rochester, NY, 14604-2701, US

(21) Appl. No.: 12/121,083

(22) Filed: May 15, 2008

Related U.S. Application Data
(60) Provisional application No. 60/938,766, filed on May 18, 2007.

(51) Int. Cl. A61K 31/498; C07D 403/12; C07C 55/20; C07C 57/03; C07C 65/10; A61P 25/00; C07C 65/05; C07C 229/36; C07C 61/29

(52) U.S. Cl. 514/249; 544/353; 562/590; 562/466; 562/477; 562/476; 562/450; 562/467

(57) ABSTRACT

A complex comprises at least an α₂-adrenergic receptor agonist and a compound that provides an opposite charge to a charge on the α₂-adrenergic receptor agonist, wherein the complex is charge neutral as a whole and has a solubility in a range from about 0.3 μg/ml to about 2.5 mg/ml in water at pH of about 7 and temperature of about 25° C. The complex is included in a composition, device, or implant for use in the neuroprotection of components of a neurological tissue to prevent progressive degeneration of such components. In particular, such a composition, device, or implant can be used to provide neuroprotection to cells and components of the optic nerve system.

### CROSS REFERENCE

This application claims the benefit of Provisional Patent Application No. 60/938,766 filed May 18, 2007.

### BACKGROUND OF THE INVENTION

The present invention relates to complexes comprising α₂-adrenergic receptor agonists and compositions comprising such complexes. In particular, the present invention relates to such compositions suitable for sustained release of α₂-adrenergic receptor agonists.

Many pathological ocular conditions, if left untreated, often lead to vision loss and eventual blindness, which are the result of progressive death of optic nerve cells. As defined by the American Academy of Opthalmology, glaucoma is an optic neuropathy with characteristic structural damage to the optic nerve, associated with progressive retinal ganglion cell death, loss of nerve fibers, and visual field loss. On the basis of its etiology, glaucoma has been classified as primary or secondary. Primary glaucoma is an independent syndrome in adults and may be classified as either chronic open-angle or chronic (acute) angle-closure. Primary open-angle glaucoma is the most commonly occurring form of glaucoma, which appears to have no attributable underlying cause. Angle-closure glaucoma usually afflicts those persons having “shallow” angles in the anterior chamber and results from the sides (or angles) of the chamber coming together and blocking aqueous outflow through the trabecular meshwork. Secondary glaucoma, as the name suggests, results from pre-existing ocular diseases such as uveitis, intraocular tumor, or enlarged cataract.

Considering all types together, glaucoma occurs in about 2 percent of all persons over the age of 40 and may be asymptomatic for years before progressing to rapid loss of vision. The underlying causes of primary glaucoma are not yet well known. An intraocular pressure (“IOP”) that is high compared to the population mean is a risk factor for the development of glaucoma. However, many individuals with high IOP do not have glaucomatous loss of vision. Conversely, there are glaucoma patients with normal IOP. Therefore, continued efforts have been devoted to elucidate the pathogenic mechanisms of glaucomatous optic nerve degeneration.

It has been postulated that optic nerve fibers are compressed by high IOP, leading to an effective physiological axotomy and problems with axonal transport. High IOP also results in compression of blood vessels supplying the optic nerve heads (“ONHs”), leading to the progressive death of retinal ganglion cells (“RGCs”). See; e.g., M. Rudzinski and H. U. Saragovi, Curr. Med. Chem.—Central Nervous System Agents, Vol. 5, 43 (2005).

In addition, there is growing evidence that other molecular mechanisms also cause direct damage to RGCs: existence of high levels of neurotoxic substances such as glutamate and nitric oxide and pro-inflammatory processes. Id. At low concentrations, NO plays a beneficial role in neurotransmission and vasodilation, while at higher concentrations, it is implicated in having a role in the pathogenesis of stroke, demyelination, and other neurodegenerative diseases. R. N. Saha and K. Pahan, Antioxidants & Redox Signaling, Vol. 8, No. 5 & 6, 929 (2006). NO has been recognized as a mediator and regulator of inflammatory responses. It possesses cytotoxic properties and is produced by immune cells, including macrophages, with the aim of assisting in the destruction of pathogenic microorganisms, but it can also have damaging effects on host tissues. NO can also react with molecular oxygen and superoxide anion to produce reactive nitrogen species that can modify various cellular functions. R. Korhonen et al., Curr. Drug Target—Inflam. & Allergy, Vol. 4, 471 (2005). Furthermore, oxidative stress, occurring not only in the trabecular meshwork (“TM”) but also in retinal cells, appears to be involved in the neuronal cell death affecting the optic nerve in primary open-angle glaucoma (“POAG”). J. Nair et al., Mutat Res., Vol. 612, No. 2, 105 (2006).

In addition, tumor necrosis factor-α (“TNF-α”), a proinflammatory cytokine, has recently been identified to be a mediator of RGC death. TNF-α and TNF-α receptor-1 are up-regulated in experimental rat models of glaucoma. In vitro studies have further identified that TNF-α-mediated RGC death involves the activation of both receptor-mediated caspase cascade and mitochondria-mediated caspase-dependent and caspase-independent components of cell death cascade. G. Tezel and X. Yang, Expt'l Eye Res., Vol. 81, 207 (2005). Moreover, TNF-α and its receptor were found in greater amounts in retina sections of glaucomatous eyes than in control eyes of age-matched normal donors. G. Tezel et al., Invest. Opthalmol. & Vis. Sci., Vol. 42, No. 8, 1787 (2001).

Regardless of the theory, glaucomatous visual field loss is a clinically recognized condition. There has been growing evidence that such vision loss results from damage to optic nerve cells.

Retinitis pigmentosa, another back-of-the-eye disease, is the term for a group of inherited diseases that affect the retina, the delicate nerve tissue composed of several cell layers that line the inside of the back of the eye and contain photoreceptor cells. These diseases are characterized by a gradual breakdown and degeneration of the photoreceptor cells, the so-called rods and cones, which result in a progressive loss of vision. It is estimated that retinitis pigmentosa affects thousands of individuals in the United States. Together, rods and cones are the cells responsible for converting light into electrical impulses that transfer messages to the retinal ganglion cells which in turn transmit the impulses through the lateral geniculate nucleus into that area of the brain where sight is perceived. Retinitis pigmentosa, therefore, affects a different retinal cell type than those affected by glaucoma. Depending on which type of photoreceptor cell is predominantly affected, the symptoms vary, and include night blindness, lost peripheral vision (also referred to as tunnel vision), and loss of the ability to discriminate color before peripheral vision is diminished. Symptoms of retinitis pigmentosa are most often recognized in adolescents and young adults, with progression of the disease usually continuing throughout the patient's life. The rate of progression and degree of visual loss are variable. As yet, there is no known cure for retinitis pigmentosa.

Age-related macular degeneration (“AMD”), another back-of-the eye disease, is a degenerative condition of the macula or central retina. It is the most common cause of vision loss in the over-50 age group. It is estimated that 50 million people worldwide suffer from AMD. Its prevalence increases with age and affects 15 percent of the population by age 55 and over 30 percent are affected by age 75. Macular degeneration can cause loss of central vision and make reading or driving impossible, but unlike glaucoma, macular degeneration does not cause complete blindness since peripheral vision is not affected. Macular degeneration is usually obvious during opthalmologic examination.

Macular degeneration is classified as either dry (non-neovascular) or wet (neovascular). In its exudative, or “wet,” form, a layer of the retina becomes elevated with fluid, causing retinal detachment and wavy vision distortions. It has recently been discovered that mutations in two genes encoding proteins in the so-called complement cascade account for most of the risk of developing AMD. This complex molecular pathway is the body's first line of defense against invading bacteria, but if overactive, the pathway can produce tissue-damaging inflammation, which underlies the vision-destroying changes that particularly strike the macula. Proteins associated with immune system activity have been found in or near drusen (yellow deposits) in eyes with the dry form of AMD. Over time, the drusen grow as they accumulate inflammatory proteins and other materials, and the inflammation persists, causing additional damage to the retina and eventual vision loss. (See; e.g., Science, Vol. 311, 1704 (2006).)

Thus, it is now known that many serious back-of-the eye pathological conditions lead to loss of vision through progressive damage to various components of the optic nerve system. Consequently, in addition to provision of treatment of the cause of the condition, it is desirable to prevent further damage to the remaining functioning cells of the optic nerve system. Recently, α₂-adrenergic receptor agonists have been noted to be neuroprotective for RGCs. See; e.g., E. WoldeMussie et al., Invest. Opthalmol. & Vis. Sci., Vol. 42, No. 12, 2849 (2001); M. P. Lafuente Lopez-Herrera et al., Expt'l Neurol., Vol. 178, 243 (2002). It has been reported that injected brimonidine and clonidine, which are among the α₂-adrenergic receptor agonists, delay the secondary degeneration of axons after a partial optic nerve crush in rats, and the neuroprotective effect could be blocked by α₂-antagonists. A. T. E. Hartwick, Optometry and Vision Science, Vol. 78, No. 2, 85 (2001) (noting E. Yoles et al., Invest. Opthalmol. Vis. Sci., Vol. 40, 65 (1999)). Brimonidine is currently formulated as brimonidine tartrate for topical administration for lowering intraocular pressure (“IOP”). Brimonidine tartrate has solubility in water of about 34 mg/ml (see US Patent Application Publication 2005/0244463 A1) and, thus, may be cleared very quickly after topical administration. Therefore, questions remain whether topical administration of soluble brimonidine tartrate would result in a therapeutically effective amount in the retina where it is needed.

Therefore, there is continued need to provide compounds and compositions comprising α₂-adrenergic receptor agonists that are present in amounts and for duration in ocular environments where they can provide effective neuroprotection to the optic nerve system. In addition, it is also desirable to provide methods for neuroprotection using such compositions.

### SUMMARY

In general, the present invention provides complexes comprising α₂-adrenergic receptor agonists and compositions comprising such complexes.

In one aspect, such complexes and compositions are used to provide neuroprotection to cells or components of a nervous system. In one embodiment, such a nervous system comprises the optic nerve system.

In another aspect, a complex of the present invention comprises at least an α₂-adrenergic receptor agonist and a compound that provides an opposite charge to a charge on the α₂-adrenergic receptor agonist at the relevant pH (such an ionized compound is also referred to herein from time to time as “counterion”). In general, a relevant pH is a range where the α₂-adrenergic receptor agonist is charged (i.e. ionized by protonation), generally a positive charge and conversely, wherein the counterion is negatively charged (i.e., ionized by deprotonation). In one embodiment, the relevant pH is the physiological pH. In another embodiment, the relevant pH is that in an ocular environment.

In still another aspect, a complex of the present invention is charge neutral as a whole.

In still another aspect, a complex of the present invention is negatively charged with a net charge ranging from −1 to −2 to −3 or −4 as a whole.

In yet another aspect, the counterion comprises an ion of carboxylic acids, sulfonic acids, or phosphonic acids.

In still another aspect, the α₂-adrenergic receptor agonist is N-(2-imidazolin-2-yl)-quinoxalinamine or a derivative thereof.

In still another aspect, a complex of the present invention has a solubility in a range from about 0.3 μg/ml to about 2.5 mg/ml in water at a relevant pH and at temperature of about 25° C.

In yet another aspect, the present invention provides a composition comprising a medium and a complex that comprises at least an α₂-adrenergic receptor agonist and a compound that provides an opposite charge to a charge on the α₂-adrenergic receptor agonist, wherein the complex is charge neutral as a whole and has a solubility in a range from about 0.3 μg/ml to about 2.5 mg/ml in water at a relevant pH and temperature of about 25° C.

In a further aspect, the present invention provides a method for neuroprotection, comprising administering to a subject in need of neuroprotection a composition that comprises a complex comprising an α₂-adrenergic receptor agonist.

Other features and advantages of the present invention will become apparent from the following detailed description and claims and the appended drawings.

### BRIEF DESCRIPTION OF THE DRAWINGS

FIG. 1 shows NMR spectrum of brimonidine free base.

FIG. 2 shows NMR spectrum pamoic acid.

FIG. 3 shows NMR spectrum of brimonidine pamoate complex.

FIG. 4 shows XRD spectra of pamoic acid (bottom curve), brimonidine free base (middle curve), and simple solid mixture of brimonidine and pamoic acid (top curve).

FIG. 5 shows XRD spectra of pamoic acid (bottom curve), brimonidine free base (top curve), and complex of brimonidine and pamoic acid (middle curve).

FIG. 6 shows XRD spectra of two different lots of complexes of brimonidine and pamoic acid prepared during scale-up experiments.

FIG. 7 shows XRD spectra of two different lots of complexes of brimonidine and 1-hydroxy-2-naphthoic acid during scale-up experiments.

FIG. 8 shows XRD spectra of two different lots of complexes of brimonidine and diatrizoic acid during scale-up experiments.

FIG. 9 shows proton NMR spectra of two different lots of complexes of brimonidine and pamoic acid prepared during scale-up experiments.

FIG. 10 shows proton NMR spectra of two different lots of complexes of brimonidine and 1-hydroxy-2-naphthoic acid during scale-up experiments.

FIG. 11 shows proton NMR spectra of two different lots of complexes of brimonidine and diatrizoic acid during scale-up experiments.

### DETAILED DESCRIPTION OF THE INVENTION

As used herein, the term “complex” means an entity formed by an association or interaction of at least two molecules, each carrying a charge at the relevant pH. In one aspect, at least two molecules of the entity carry opposite charges. In another aspect, the complex is charge neutral as a whole.

As used herein, the term “neuroprotection” means the rescue of at least some cells or components of a nervous system that are not directly damaged by the primary cause of a disease or injury, but would otherwise undergo secondary degeneration without therapeutic intervention. In one aspect, neuroprotection can lead to preservation of the physiological function of these cells or components. In one aspect, such a nervous system is the optic nerve system. The cells or components of the optic nerve system include those being involved or assisting in conversion of photon to neurological signal and the transmission thereof from the retina to the brain for processing. Thus, the main cells or components of the optic nerve system include, but are not limited to, pigment epithelial cells, photoreceptor cells (rod and cone cells), bipolar cells, horizontal cells, amacrine cells, interplexiform cells, ganglion cells, support cells to ganglion cells, and optic nerve fibers.

As used herein, the term “lower alkyl” or “lower alkyl group” means a C₁-C₁₀ alkyl group. The term “lower alkoxy” or “lower alkoxy group” means C₁-C₁₀ alkoxy group.

In general, the present invention provides complexes comprising α₂-adrenergic receptor agonists and compositions comprising such complexes.

In one aspect, such complexes and compositions are used to provide neuroprotection to cells or components of a nervous system. In one embodiment, such a nervous system comprises the optic nerve system. In another embodiment, the cells or components of the optic nerve system that can derive therapeutic benefits from a composition of the present invention are selected from the group consisting of pigment epithelial cells, photoreceptor cells (rod and cone cells), bipolar cells, horizontal cells, amacrine cells, interplexiform cells, ganglion cells, support cells to ganglion cells, optic nerve fibers, and combinations thereof.

In another aspect, a complex of the present invention comprises at least an α₂-adrenergic receptor agonist and a compound that provides an opposite charge to a charge on the α₂-adrenergic receptor agonist at the relevant pH (such a charged compound is also referred to herein from time to time as “counterion”). In one embodiment, the relevant pH is a range from about 7 to about 7.5. In another embodiment, the relevant pH is the physiological pH. In still another embodiment, the relevant pH is that in an ocular environment. In yet another embodiment, the relevant pH is about 7.4.

In still another aspect, a complex of the present invention is charge neutral as a whole.

In still another aspect, a complex of the present invention can be negatively charged ranging from −1 to −2 to −3 to −4 as a whole.

In yet another aspect, the counterion comprises an ion of carboxylic acids, sulfonic acids, or phosphonic acids. In one embodiment, the counterion comprises an ion other than that of a fatty acid. In another embodiment, the counterion comprises an ion of pharmaceutically acceptable carboxylic acids other than fatty acids, pharmaceutically acceptable sulfonic acids, or pharmaceutically acceptable phosphonic acids.

In still another aspect, the counterion comprises an ion of pharmaceutically acceptable carboxylic acids other than fatty acids, pharmaceutically acceptable sulfonic acids, or pharmaceutically acceptable phosphonic acids and has one, two, three, four, five, or more negative charges at pH in a range from about 7 to about 7.5.

In yet another aspect, the counter ion comprises an ion of an organic acid selected from the group consisting of pamoic acid, sebacic acid, hippuric acid, capric acid, mandelic acid, (S)-(+)-2-(6-methoxy-2-naphthyl)propionic acid (naproxen), dichloroacetic acid, adipic acid, 4-acetamidobenzoic acid, cinnamic acid, dodecylsulfuric acid, salicylic acid, gentisic acid, naphthalene-2-sulfonic acid, 1-hydroxy-2-naphthoic acid, docosahexaenoic acid (“DHA”), arachidonic acid, eicosenoic acid, cholesteric acid, taurocholic acid, taurodeoxycholic acid, taurochenodeoxycholic acid, glycocholic acid, glycochenodeoxycholic acid, diatrizoic acid (iodamide), iobenzamic acid (N-(3-amino-2,4,6-triiodobenzoyl)N-phenyl-β-alanine), iocarmic acid (3,3′-((1,6-dioxo-1,6-hexanediyl)diimino)bis(2,4,6-triiodo-5-(methylamino)carbonyl)benzoic acid), iocetamic acid N-acetyl-N-(3-amino-2,4,6-triiodophenyl)amino-isobutyric acid), iodipamide (3,3′-(adipoyidiimino)bis(2,4,6-triiododenzoic acid)), iodoalphionic acid, iodobenzoic acid, ioglycamic acid (3,3′-(oxybis((1-oxo-2,1-ethanediyl)imino))bis(2,4,6-triiodobenzoic acid)), iomeglamic acid (5-((3-amino-2,4,6-triiodophenyl)methylamino)-5-oxopentanoic acid), iopanoic acid (3-amino-α-ethyl-2,4,6-triiodo-benzenepropanoic acid), iophenoxic acid (α-ethyl-3-hydroxy-2,4,6-triiodobenznepropanoic acid), iopronic acid (2-((3-acetamino-2,4,6-triiodophenoxy)-2-ethoxy)methylbutyric acid), iothalamic acid (3-(acetylamino)-2,4,6-triiodo-5-((methylamino)carbonyl-benzoic acid), ioxaglic acid (3-((((3-(acetylmethylamino)-2,4,6-triiodo-5-((methylamino)carbonyl)benzoyl)amino)acetyl)amino)-5-(((2-hydroxyethyl)amino)carbonyl)-2,4,6-triiodobenzoic acid), ipodate (β-(3-dimethylaminomethyleneamino-2,4,6-triiodophenyl)propionic acid), ethylenediaminetetraacetic acid (“EDTA”), diethylenetriaminepentaacetic acid (“DTPA”), 1,4,7,10-tetraazacyclododecane-N,N′,N″,N′″-tetraacetic acid (“DOTA”), benzyloxypropionictetraacetic acid (“BOPTA”), triethylenetetraminehexaacetic acid (“TTHA”), 1,2-diaminocyclohexane-N,N,N′,N′-tetraacetic acid, N-(2-hydroxyethyl)ethylenediaminetriacetic acid, nitrilotriacetic acid, ethylene-bis(oxyethylenenitrilo)tetraacetic acid (“EGTA”), 1,4,7,10-tetraazacyclododecane-N,N′,N″-triacetic acid (“DO3A”), 1,4,7-tris(carboxymethyl)-10-(2′-hydroxy)propyl)-1,4,7,10-tetraazocyclodecane (“HP-DO3A”), 1,4,7-triazacyclononane-N,N′,N-triacetic acid (“NOTA”), 1,4,8,11-tetraazacyclotetradecane-N,N′,N″,N′″-tetraacetic acid (“TETA”), combinations thereof, and mixtures thereof. In one embodiment, the counter ion comprises an ion of an organic acid selected from the group consisting of pamoic acid, sebacic acid, hippuric acid, mandelic acid, (S)-(+)-2-(6-methoxy-2-naphthyl)propionic acid (naproxen), dichloroacetic acid, adipic acid, 4-acetamidobenzoic acid, cinnamic acid, dodecylsulfuric acid, salicylic acid, gentisic acid, naphthalene-2-sulfonic acid, 1-hydroxy-2-naphthoic acid, combinations thereof, and mixture thereof. In another embodiment, the counter ion comprises an ion of an organic acid selected from the group consisting of cholesteric acid, taurocholic acid, taurodeoxycholic acid, taurochenodeoxycholic acid, glycocholic acid, glycochenodeoxycholic acid, combinations thereof, and mixtures thereof. In still another embodiment, the counter ion comprises an ion of an organic acid selected from the group consisting of diatrizoic acid (iodamide), iobenzamic acid, iocarmic acid, iocetamic acid, iodipamide (3,3′-(adipoyldiimino)bis(2,4,6-triiododenzoic acid)), iodoalphionic acid, iodobenzoic acid, ioglycamic acid, iomeglamic acid, iopanoic acid, iophenoxic acid, iopronic acid, iothalamic acid, ioxaglic acid, ipodate (β-(3-dimethylaminomethyleneamino-2,4,6-triiodophenyl)propionic acid, ethylenediaminetetraacetic acid (“EDTA”), diethylenetriaminepentaacetic acid (“DTPA”), 1,4,7,10-tetraazacyclododecane-N,N′,N″,N′″-tetraacetic acid (“DOTA”), benzyloxypropionictetraacetic acid (“BOPTA”), triethylenetetraminehexaacetic acid (“TTHA”), 1,2-diaminocyclohexane-N,N,N′,N′-tetraacetic acid, N-(2-hydroxyethyl)ethylenediaminetriacetic acid, nitrilotriacetic acid, ethylene-bis(oxyethylenenitrilo)tetraacetic acid (“EGTA”), 1,4,7,10-tetraazacyclododecane-N,N′,N″-triacetic acid (“DO3A”), 1,4,7-tris(carboxymethyl)-10-(2′-hydroxy)propyl)-1,4,7,10-tetraazocyclodecane (“HP-DO3A”), 1,4,7-triazacyclononane-N,N′,N-triacetic acid (“NOTA”), 1,4,8,11-tetraazacyclotetradecane-N,N′,N″,N′″-tetraacetic acid (“TETA”), combinations thereof, and mixtures thereof.

It may be advantageous to provide a counterion that has two, three, four, five, six, or more charges to increase the amount of the α₂-adrenergic receptor agonist in the composition that is delivered to the site of damaged tissue.

In certain embodiments, the counterion of such a complex excludes polyanionic polymers. In certain other embodiments, the counterion of such a complex excludes synthetic polyanionic polymers.

In still another aspect, a complex of the present invention has a solubility in a range from about 0.3 μg/ml to about 2.5 mg/ml in water at pH of about 7. A concentration of at least about 0.3 μg/ml of the complex near the site of the damaged tissue is believed adequately to provide therapeutic value for neuroprotection. In one embodiment, a complex of the present invention has a solubility in a range from about 0.3 μg/ml to about 2 mg/ml in water at pH of about 7 and at a temperature of about 25° C. Alternatively, a complex of the present invention has a solubility in a range from about 0.3 μg/ml to about 1.5 mg/ml in water at pH of about 7 and at a temperature of about 25° C. (or from about 0.3 μg/ml to about 1 mg/ml, or from about 0.5 μg/ml to about 1 mg/ml, or from about 1 μg/ml to about 1 mg/ml, or from about 10 μg/ml to about 2 mg/ml, or from about 10 μg/ml to about 0.5 mg/ml, or from about 10 μg/ml to about 100 μg/ml, in water at pH of about 7 and at a temperature of about 25° C.).

In still another aspect, a complex of the present invention has a solubility in a range from about 0.3 μg/ml to about 2.5 mg/ml in water at a relevant pH. A concentration of at least about 0.3 μg/ml of the complex near the site of the damaged tissue is believed adequately to provide therapeutic value for neuroprotection. In one embodiment, a complex of the present invention has a solubility in a range from about 0.3 μg/ml to about 2 mg/ml in water at a relevant pH and at a temperature of about 25° C. Alternatively, a complex of the present invention has a solubility in a range from about 0.3 μg/ml to about 1.5 mg/ml in water at a relevant pH and at a temperature of about 25° C. (or from about 0.3 μg/ml to about 1 mg/ml, or from about 0.5 μg/ml to about 1 mg/ml, or from about 1 μg/ml to about 1 mg/ml, or from about 10 μg/ml to about 2 mg/ml, or from about 10 μg/ml to about 1 mg/ml, or from about 10 μg/ml to about 0.5 mg/ml, or from about 10 μg/ml to about 100 μg/ml, in water at a relevant pH and at a temperature of about 25° C.).

In still another aspect, the α₂-adrenergic receptor agonist useful in this invention include quinoxalines and derivatives thereof, including brimonidine; imino-imidazolines, including clonidine, apraclonidine; imidazolines, including naphazoline, xymetazoline, tetrahydrozoline, and tramazoline; imidazoles, including detomidine, medetomidine, and dexmedetomidine; azepines, including B-HT 920 (6-allyl-2-amino-5,6,7,8-tetrahydro-4H-thiazolo[4,5-d]-azepine and B-HT 933 (6-ethyl-2-amino-5,6,7,8-tetrahydro-4H-oxazolo[4,5-d]-azepine) available from Sigma Aldrich; thiazines, including xylazine; oxazolines, including rilmenidine; guanidines, including guanabenz and guanfacine; catecholamines; and derivatives thereof.

In yet another aspect, the α₂-adrenergic receptor agonist comprises or is quinoxalines or derivatives thereof. Non-limiting examples of suitable quinoxalines and derivatives thereof and methods for their preparation are disclosed in U.S. Pat. Nos. 5,703,077 and 3,890,319, which are incorporated herein by reference.

In still another aspect, the α₂-adrenergic receptor agonist comprises or is N-(2-imidazolin-2-yl)-quinoxalinamine or a derivative thereof.

In yet another aspect, the α₂-adrenergic receptor agonist has Formula I

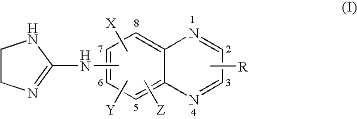

wherein the 2-imidazolin-2-ylamino group is attached to the 5-, 6-, 7-, or 8-position of the quinoxaline nucleus; X, Y, and Z are attached to the remaining 5-, 6-, 7-, and 8-positions; each of X, Y, and Z is independently selected from the group consisting of hydrogen, halogen (such as chlorine, bromine, or iodine; preferably, bromine), lower alkyl, lower alkoxy, and trifluoromethyl; and R comprises a substituent attached to the 2- or 3-position of the quinoxaline nucleus and is selected from the group consisting of hydrogen, lower alkyl, and lower alkoxy. In one embodiment, each of the lower alkyl and lower alkoxy groups comprises one to five carbon atoms. Alternatively, each of the lower alkyl and lower alkoxy groups comprises one to three carbon atoms.

In another aspect, the α₂-adrenergic receptor agonist has Formula II (5-bromo-N-(2-imidazolin-2-yl)-6-quinoxalinamine, 5-bromo-N-(4,5-dihydro-1H-imidazol-2-yl)-6-quinoxalinamine, or brimonidine).

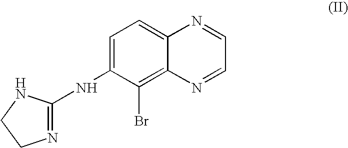

In still another aspect, the present invention provides a pharmaceutical composition comprising a complex that comprises at least an α₂-adrenergic receptor agonist and a counterion. The pharmaceutical composition can be used to provide neuroprotection to cells and components of a nervous system. In another embodiment, the nervous system comprises the optic nerve system.

In another aspect, the complex included in the pharmaceutical composition has solubility in a range from about 0.3 μg/ml to about 2.5 mg/ml in water at pH of about 7 and at a temperature of about 25° C. A concentration of at least about 0.3 μg/ml of the complex near the site of the damaged tissue is believed adequately to provide therapeutic value for neuroprotection. In one embodiment, a complex of the present invention has solubility in a range from about 0.3 μg/ml to about 2 mg/ml in water at pH of about 7 and at temperature of about 25° C. Alternatively, a complex of the present invention has a solubility in a range from about 0.3 μg/ml to about 1.5 mg/ml in water at pH of about 7 and at temperature of about 25° C. (or from about 0.3 μg/ml to about 1 mg/ml, or from about 0.5 μg/ml to about 1 mg/ml, or from about 1 μg/ml to about 1 mg/ml, or from about 10 μg/ml to about 2 mg/ml, or from about 10 μg/ml to about 1 mg/ml, or from about 10 μg/ml to about 0.5 mg/ml, or from about 10 μg/ml to about 100 μg/ml, in water at pH of about 7 in water at pH of about 7 and at temperature of about 25° C.).

In still another aspect, the complex is present in the composition in an amount in a range from about 0.0001 to about 10 percent (weight by volume). As used herein, the phrase “1 percent (weight by volume),” for example, means 1 gram in 100 ml of the composition. In one embodiment, the complex is present in the composition in an amount in a range from about 0.0005 to about 5 percent (weight by volume), or alternatively, from about 0.001 to about 1, or from about 0.001 to about 0.5, or from about 0.002 to about 0.2, or from about 0.005 to about 0.1 percent (weight by volume).

In one embodiment, a composition of the present invention is in a form of a suspension or dispersion. In another embodiment, the suspension or dispersion is based on an aqueous solution. For example, a composition of the present invention can comprise micrometer- or nanometer-sized particles of the complex suspended or dispersed in sterile saline solution. In another embodiment, the suspension or dispersion is based on a hydrophobic medium. For example, the micrometer- or nanometer-sized particles of the complex can be suspended in a hydrophobic solvent e.g., silicone oil, mineral oil, or any other suitable nonaqueous medium for delivery to the eye. In still another embodiment, the micrometer- or nanometer-sized particles of the complex can be coated with a physiologically acceptable surfactant (non-limiting examples are disclosed below), then the coated particles are dispersed in a liquid medium. The coating can keep the particles in a suspension. Such a liquid medium can be selected to produce a sustained-release suspension. For example, the liquid medium can be one that is sparingly soluble in the ocular environment into which the suspension is administered. In still another embodiment, the complex is suspended or dispersed in a hydrophobic medium, such as an oil. In still another embodiment, such a medium comprises an emulsion of a hydrophobic material and water. In still another embodiment, the insoluble complex disclosed herein can be dosed by any normal drug delivery vehicle including but not limited to suspension in a liposome formulation (both within and outside the liposome wall or strictly outside the liposome core), in the continuous phase of an emulsion or microemulsion, in the oil phase of the emulsion, or in a micellar solution using either charged or uncharged surfactants. A micellar solution wherein the surfactant is both the micelle forming agent and the anion of the complex disclosed herein would be preferable.

In another aspect, a composition of the present invention can further comprise a non-ionic surfactant, such as polysorbates (such as polysorbate 80 (polyoxyethylene sorbitan monooleate), polysorbate 60 (polyoxyethylene sorbitan monostearate), polysorbate 20 (polyoxyethylene sorbitan monolaurate), commonly known by their trade names of Tween® 80, Tween® 60, Tween® 20), poloxamers (synthetic block polymers of ethylene oxide and propylene oxide, such as those commonly known by their trade names of Pluronic®; e.g., Pluronic® F127 or Pluronic® F108)), or poloxamines (synthetic block polymers of ethylene oxide and propylene oxide attached to ethylene diamine, such as those commonly known by their trade names of Tetronic®; e.g., Tetronic® 1508 or Tetronic® 908, etc., other nonionic surfactants such as Brij®, Myrj®, and long chain fatty alcohols (i.e., oleyl alcohol, stearyl alcohol, myristyl alcohol, docosohexanoyl alcohol, etc.) with carbon chains having about 12 or more carbon atoms (e.g., such as from about 12 to about 24 carbon atoms). Such compounds are delineated in Martindale, 34^{th} ed., pp. 1411-1416 (Martindale, “The Complete Drug Reference,” S. C. Sweetman (Ed.), Pharmaceutical Press, London, 2005) and in Remington, “The Science and Practice of Pharmacy,” 21^{st} Ed., p. 291 and the contents of chapter 22, Lippincott Williams & Wilkins, New York, 2006); the contents of these sections are incorporated herein by reference. The concentration of a non-ionic surfactant, when present, in a composition of the present invention can be in the range from about 0.001 to about 5 weight percent (or alternatively, from about 0.01 to about 4, or from about 0.01 to about 2, or from about 0.01 to about 1, or from about 0.01 to about 0.5 weight percent). Any of these surfactants also can be used to coat micrometer- or nanometer-sized particles, as disclosed above.

In addition, a composition of the present invention can include additives such as buffers, diluents, carriers, adjuvants, or other excipients. Any pharmacologically acceptable buffer suitable for application to the eye may be used. Other agents may be employed in the composition for a variety of purposes. For example, buffering agents, preservatives, co-solvents, oils, humectants, emollients, stabilizers, or antioxidants may be employed.

Water-soluble preservatives which may be employed include sodium bisulfite, sodium bisulfate, sodium thiosulfate, benzalkonium chloride, chlorobutanol, thimerosal, ethyl alcohol, methylparaben, polyvinyl alcohol, benzyl alcohol, and phenylethyl alcohol. These agents may be present in individual amounts of from about 0.001 to about 5 percent by weight (preferably, about 0.01 to about 2 percent by weight).

Suitable water-soluble buffering agents that may be employed are sodium carbonate, sodium borate, sodium phosphate, sodium acetate, sodium bicarbonate, etc., as approved by the United States Food and Drug Administration (“US FDA”) for the desired route of administration. These agents may be present in amounts sufficient to maintain a pH of the system of between about 6 and about 8. As such, the buffering agent may be as much as about 5% on a weight to weight basis of the total composition. Electrolytes such as, but not limited to, sodium chloride and potassium chloride may also be included in the formulation. Physiologically acceptable buffers include, but are not limited to, a phosphate buffer or a Tris-HCl buffer (comprising tris(hydroxymethyl)aminomethane and HCl). For example, a Tris-HCl buffer having pH of 7.4 comprises 3 g/l of tris(hydroxymethyl)aminomethane and 0.76 g/l of HCl. In yet another aspect, the buffer is 10× phosphate buffer saline (“PBS”) or 5×PBS solution.

Other buffers also may be found suitable or desirable in some circumstances, such as buffers based on HEPES (N-{2-hydroxyethyl}peperazine-N′-{2-ethanesulfonic acid}) having pKₐ of 7.5 at 25° C. and pH in the range of about 6.8-8.2; BES (N,N-bis{2-hydroxyethyl}2-aminoethanesulfonic acid) having pKₐ of 7.1 at 25° C. and pH in the range of about 6.4-7.8; MOPS (3-{N-morpholino}propanesulfonic acid) having pKₐ of 7.2 at 25° C. and pH in the range of about 6.5-7.9; TES (N-tris{hydroxymethyl}-methyl-2-aminoethanesulfonic acid) having pKₐ of 7.4 at 25° C. and pH in the range of about 6.8-8.2; MOBS (4-{N-morpholino}butanesulfonic acid) having pKₐ of 7.6 at 25° C. and pH in the range of about 6.9-8.3; DIPSO (3-(N,N-bis{2-hydroxyethyl}amino)-2-hydroxypropane)) having pKₐ of 7.52 at 25° C. and pH in the range of about 7-8.2; TAPSO (2-hydroxy-3{tris(hydroxymethyl)methylamino}1-propanesulfonic acid)) having pKₐ of 7.61 at 25° C. and pH in the range of about 7-8.2; TAPS ({(2-hydroxy-1,1-bis(hydroxymethyl)ethyl)amino}-1-propanesulfonic acid)) having pKₐ of 8.4 at 25° C. and pH in the range of about 7.7-9.1; TABS (N-tris(hydroxymethyl)methyl-4-aminobutanesulfonic acid) having pKₐ of 8.9 at 25° C. and pH in the range of about 8.2-9.6; AMPSO (N-(1,1-dimethyl-2-hydroxyethyl)-3-amino-2-hydroxypropanesulfonic acid)) having pKₐ of 9.0 at 25° C. and pH in the range of about 8.3-9.7; CHES (2-cyclohexylamino)ethanesulfonic acid) having pKₐ of 9.5 at 25° C. and pH in the range of about 8.6-10.0; CAPSO (3-(cyclohexylamino)-2-hydroxy-1-propanesulfonic acid) having pKₐ of 9.6 at 25° C. and pH in the range of about 8.9-10.3; or CAPS (3-(cyclohexylamino)-1-propane sulfonic acid) having pKₐ of 10.4 at 25° C. and pH in the range of about 9.7-11.1.

In one aspect, the composition has a relevant pH, wherein a relevant pH is a range where the α₂-adrenergic receptor agonist is charged (i.e. ionized by protonation), generally a positive charge and conversely, wherein the counterion is negatively charged (i.e., ionized by deprotonation).

In one aspect, the composition has a pH of about 7. Alternatively, the composition has a pH in a range from about 7 to about 7.5.

In another aspect, the composition has a pH of about 7.4.

In yet another aspect, a composition also can comprise a viscosity-modifying compound designed to facilitate the administration of the composition into the subject or to promote the bioavailability in the subject. In still another aspect, the viscosity-modifying compound may be chosen so that the composition is not readily dispersed after being administered into an ocular environment (such as the ocular surface, conjunctiva, or vitreous). Such compounds may enhance the viscosity of the composition, and include, but are not limited to: monomeric polyols, such as, glycerol, propylene glycol, ethylene glycol; polymeric polyols, such as, polyethylene glycol; various polymers of the cellulose family, such as hydroxypropylmethyl cellulose (“HPMC”), carboxymethyl cellulose (“CMC”) sodium, hydroxypropyl cellulose (“HPC”); polysaccharides, such as hyaluronic acid and its salts, chondroitin sulfate and its salts, dextrans, such as, dextran 70; water soluble proteins, such as gelatin; vinyl polymers, such as, polyvinyl alcohol, polyvinylpyrrolidone, povidone; carbomers, such as carbomer 934P, carbomer 941, carbomer 940, or carbomer 974P; and acrylic acid polymers. In general, a desired viscosity can be in the range from about 1 to about 400 centipoises (“cp” or mPa·s).

In another aspect, the present invention provides a method for producing a composition comprising a complex that comprises at least an α₂-adrenergic receptor agonist and a counterion, the method comprising: (a) providing said complex that has a solubility in a medium and a portion of which complex remains in a solid phase for a period longer than one day after said complex has been in contact with said medium; and (b) dispersing an amount of said complex in a sufficient amount of said medium to produce said composition to achieve a predetermined concentration of said complex in said medium. Alternatively, a portion of the complex remains in a solid phase for a period longer than 2 days, or 1 week, or 1 month, or 2 months, or 3 months, or 4 months, or 5 months, or 6 months after said complex has been in contact with said medium. In one embodiment, the method can optionally include a step of reducing the size of the complex before dispersing such complex in the medium.

In still another aspect, the present invention provides a method for producing a composition comprising a complex that comprises at least an α₂-adrenergic receptor agonist and a counterion, the method comprising: (a) providing said at least an α₂-adrenergic receptor agonist and a compound that is ionizable to said counterion; (b) mixing said at least an α₂-adrenergic receptor agonist and said compound to produce the complex; (c) adjusting a pH of a mixture of said at least an α₂-adrenergic receptor agonist and said compound to a pH such that a precipitate of the complex forms; (d) recovering the precipitate of the complex; and (e) dispersing the precipitate in an amount of a medium to produce said composition to achieve a predetermined concentration of said complex in said medium. In one embodiment, a portion of the complex remains in a solid phase for a period longer than one day after said complex has been in contact with said medium. In one embodiment, the method can optionally include a step of reducing the size of the precipitate after its recovery and before dispersing such precipitate having reduced sized in the medium.

In still another aspect, said at least an α₂-adrenergic receptor agonist has Formula I or II.

In another aspect, a formulation comprising a complex that comprises at least an α₂-adrenergic receptor agonist and a counterion is prepared for topical administration, periocular injection, or intravitreal injection. An injectable intravitreal formulation can desirably comprise a carrier that provides a sustained-release of the active ingredients, such as for a period longer than about one day, or one week, or longer than about 1, 2, 3, 4, 5, or 6 months. In certain embodiments, the sustained-release formulation desirably comprises a carrier that is insoluble or only sparingly soluble in an ocular environment (such as the ocular surface, conjunctiva, or vitreous). Such a carrier can be an oil-based liquid, emulsion, gel, or semisolid. Non-limiting examples of oil-based liquids include castor oil, peanut oil, olive oil, coconut oil, sesame oil, cottonseed oil, corn oil, sunflower oil, fish-liver oil, arachis oil, and liquid paraffin.

In one aspect, a composition of the present invention can be administered into a subject in need of neuroprotection at one time or over a series of treatments. A composition of the present invention may be administered locally; e.g., intravitreally by intrabulbar injection for ocular neuroprotection, or by intrathecal or epidural administration for spinal protection. Many of the compositions of the invention can be administered systemically; e.g., orally, or intravenously, or by intramuscular injection. In addition, compositions for protection of the retina and optic nerve that are capable of passing through the cornea and achieving sufficient concentration in the vitreous humor (such as a concentration disclosed herein above) may also be administered topically to the eye. In one embodiment, the neuroprotection can prevent progressive damage to cells or components of the optic nerve, which damage results from glaucoma, retinitis pigmentosa, AMD, diabetic retinopathy, diabetic macular edema, or other back-of-the-eye diseases.

In one embodiment, a composition of the present invention can be injected intravitreally, for example through the pars plana of the ciliary body, to treat or prevent glaucoma or progression thereof, or to provide neuroprotection to the optic nerve system, using a fine-gauge needle, such as 25-30 gauge. Typically, an amount from about 25 μl to about 100 μl of a composition comprising a complex that comprises at least an α₂-adrenergic receptor agonist and a counterion is administered into a patient. A concentration of such a complex is selected from the ranges disclosed above.

In still another aspect, a complex that comprises at least an α₂-adrenergic receptor agonist and a counterion is incorporated into an ophthalmic device or system that comprises a biodegradable material, and the device is injected or implanted into a subject to provide a long-term (e.g., longer than about 1 week, or longer than about 1, 2, 3, 4, 5, or 6 months) treatment or prevention of glaucoma or progression thereof, or to provide neuroprotection to the optic nerve system. In some embodiments, the ophthalmic device or system can comprise a semipermeable membrane that allows the complex to diffuse therethrough at a controlled rate. In still some other embodiments, such a controlled rate provides a supply of the complex over an extended period of time at or near the site of desired treatment. Such a device system may be injected or implanted by a skilled physician in the subject's ocular or periocular tissue.

Some compositions of the present invention are disclosed in the examples below. It should be understood that the proportions of the listed ingredients may be adjusted for specific circumstances.

### EXAMPLE 1

The ingredients listed in Table 1 are mixed for at least 15 minutes. The pH of the combined mixture is adjusted to 7-7.5 using 1 N NaOH or 1 N HCl solution to yield a composition of the present invention.

TABLE 1

| Ingredient | Amount | |
|---|---|---|
| Carbopol 934P NF | 0.25 | g |
| Purified water | 99.75 | g |
| Propylene glycol | 5 | g |
| EDTA | 0.1 | mg |
| Complex of brimonidine and pamoic | 100 | mg |
| acid | | |

### EXAMPLE 2

The ingredients listed in Table 2 are mixed together for at least 15 minutes. The pH of the mixture is adjusted to 7-7.5 using 1 N NaOH or 1 N HCl solution to yield a composition of the present invention.

TABLE 2

| | Amount (% by weight, except |
|---|---|
| Ingredient | where “ppm” is indicated) |
| Povidone | 1.5 |
| HAP (30%) | 0.05 |
| Glycerin | 3 |
| Propylene glycol | 3 |
| Complex of brimonidine and hippuric | 0.5 |
| acid | |
| Alexidine 2HCl | 1-2 ppm |
| Purified water | q.s. to 100 |

Note: “HAP” denotes hydroxyalkyl phosphonates, such as those known under the trade name Dequest®. HAPs can be used as chelating agents and have been shown to inhibit bacterial and fungal cell replication.

### EXAMPLE 3

The ingredients listed in Table 3 are mixed together for at least 15 minutes. The pH of the mixture is adjusted to 7-7.5 using 1 N NaOH or 1 N HCl solution to yield a composition of the present invention.

TABLE 3

| | Amount (% by weight, except |
|---|---|
| Ingredient | where “ppm” is indicated) |
| CMC (MV) | 0.5 |
| HAP (30%) | 0.05 |
| Glycerin | 3 |
| Propylene glycol | 3 |
| Complex of brimonidine and EDTA | 0.25 |
| Tyloxapol (a surfactant) | 0.25 |
| Alexidine 2HCl | 1-2 ppm |
| Sunflower oil | q.s. to 100 |

### EXAMPLE 4

The ingredients listed in Table 4 are mixed together for at least 15 minutes. The pH of the mixture is adjusted to 7-7.5 using 1 N NaOH or 1 N HCl solution to yield a composition of the present invention.

TABLE 4

| | Amount (% by weight, except |
|---|---|
| Ingredient | where “ppm” is indicated) |
| CMC (MV) | 0.5 |
| Glycerin | 3 |
| Propylene glycol | 3 |
| Complex of brimonidine and DTPA | 0.3 |
| Polysorbate 80 ® (a surfactant) | 0.25 |
| Alexidine 2HCl | 1-2 ppm |
| Purified water | q.s. to 100 |

### EXAMPLE 5

The ingredients listed in Table 5 are mixed together for at least 15 minutes. The pH of the mixture is adjusted to 7-7.5 using 1 N NaOH or 1 N HCl solution to yield a composition of the present invention.

TABLE 5

| | Amount (% by weight, except |
|---|---|
| Ingredient | where “ppm” is indicated) |
| CMC (MV) | 0.5 |
| Glycerin | 3 |
| Propylene glycol | 3 |
| Complex of brimonidine and sebacic | 0.5 |
| acid | |
| Tyloxapol (a surfactant) | 0.25 |
| Alexidine 2HCl | 1-2 ppm |
| Corn oil | q.s. to 100 |

### EXAMPLE 6

The ingredients listed in Table 6 are mixed together for at least 15 minutes. The pH of the mixture is adjusted to 7-7.5 using 1 N NaOH or 1 N HCl solution to yield a composition of the present invention.

TABLE 6

| | Amount (% by weight, except |
|---|---|
| Ingredient | where “ppm” is indicated) |
| CMC (MV) | 0.5 |
| Glycerin | 3 |
| Propylene glycol | 3 |
| Complex of α₂-adrenergic receptor | 0.75 |
| agonist having Formula I and naproxen | |
| Tyloxapol (a surfactant) | 0.25 |
| Alexidine 2HCl | 1-2 ppm |
| Purified water | q.s. to 100 |

### EXAMPLE 7

The ingredients listed in Table 7 are mixed together for at least 15 minutes. The pH of the mixture is adjusted to 7-7.5 using 1 N NaOH or 1 N HCl solution to yield a composition of the present invention.

TABLE 7

| | Amount (% by weight, except |
|---|---|
| Ingredient | where “ppm” is indicated) |
| HPMC | 0.5 |
| Glycerin | 3 |
| Propylene glycol | 3 |
| Complex of B-HT 933 (6-ethyl-5,6,7,8- | 0.6 |
| tetrahydro-4H-oxazolo[4,5-d]azepin-2- | |
| amine) and salicylic acid | |
| Tyloxapol (a surfactant) | 0.25 |
| Alexidine 2HCl | 1-2 ppm |
| Purified water | q.s. to 100 |

### EXAMPLE 8

The ingredients listed in Table 8 are mixed together for at least 15 minutes. The pH of the mixture is adjusted to 7-7.5 using 1 N NaOH or 1 N HCl solution to yield a composition of the present invention.

TABLE 8

| | Amount (% by weight, except |
|---|---|
| Ingredient | where “ppm” is indicated) |
| HPC | 0.5 |
| Glycerin | 3 |
| Propylene glycol | 3 |
| Complex of 5-chloro-N-(2-imidazolin-2- | 1 |
| yl)-6-quinoxalinamine and gentisic acid | |
| Pluronic ® F127 (a surfactant) | 0.25 |
| Alexidine 2HCl | 1-2 ppm |
| Purified water | q.s. to 100 |

Alternatively, purified water may be substituted with an oil, such as fish-liver oil, peanut oil, sesame oil, coconut oil, sunflower oil, corn oil, or olive oil to produce an oil-based formulation comprising an α₂-adrenergic receptor agonist.

Benefits of complexes or compositions of the present invention for neuroprotection can be determined, judged, estimated, or inferred by conducting assays and measurements, for example, to determine: (1) the protection of nerve cells from glutamate induced toxicity; and/or (2) the neural protection in a nerve crush model of mechanical injury. Non-limiting examples of such assays and measurements are disclosed in U.S. Pat. No. 6,194,415, which is incorporated herein by reference.

### EXAMPLE 9

### Preparation of Brimonidine Pamoate Complex

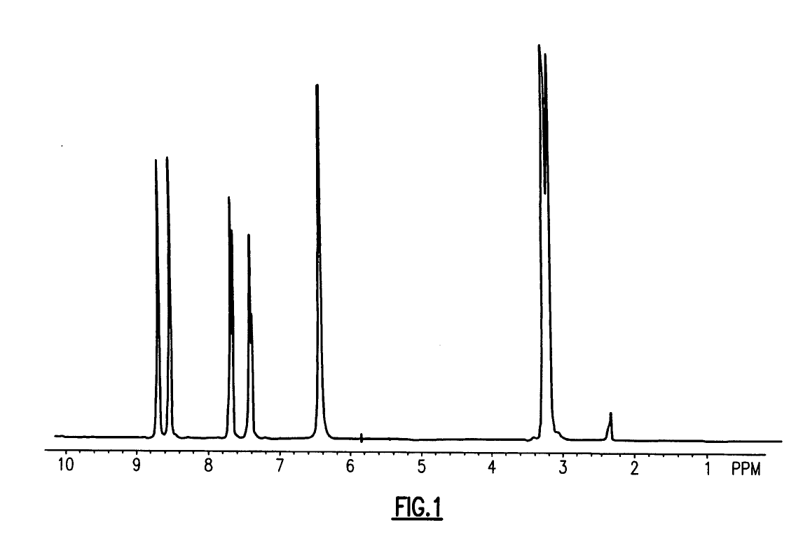

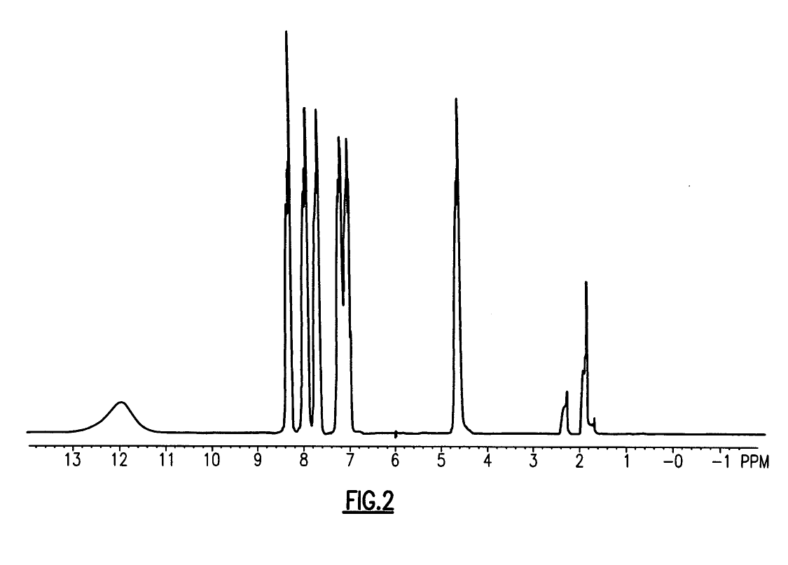

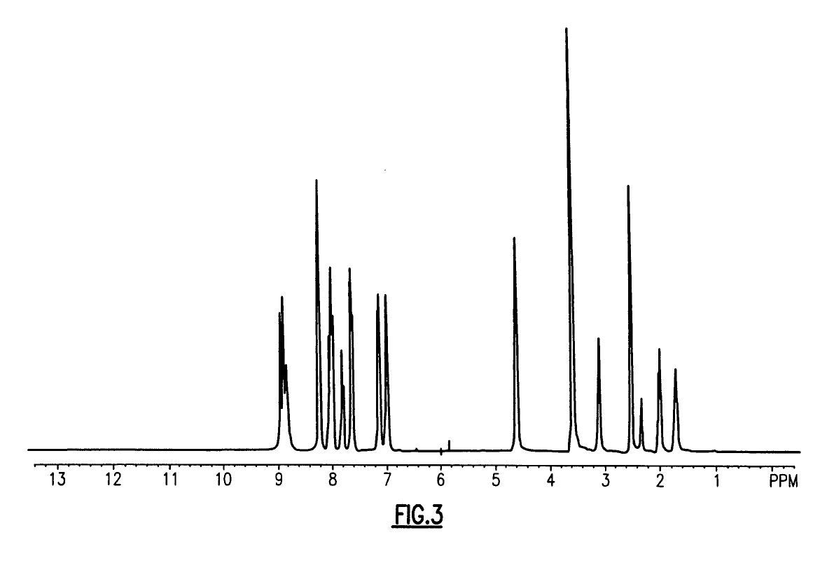

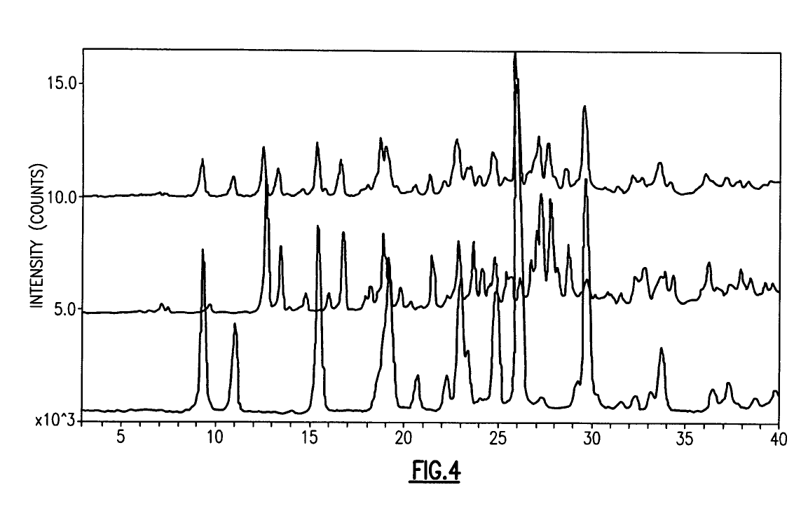

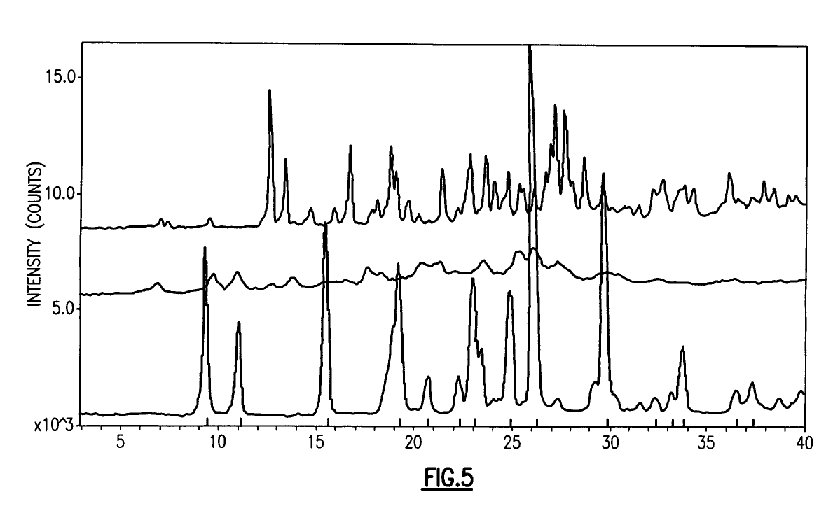

Brimonidine and pamoic acid, at a molar ratio of 1:1, were mixed and dispersed in N-methylpyrrolidone (“NMP”). The solution of brimonidine and pamoic acid in NMP was heated while stirring to dissolve brimonidine (the final temperature, typically less than 70° C., was chosen so as not to lose significant amount of solvent). Water, as an anti-solvent, was added until the solution began to become cloudy, indicating that the complex started to precipitate. The precipitation was allow to proceed; for example, for several hours or overnight. The precipitated was filtered under vacuum, and dry solid comprising the brimonidine pamoate complex was recovered. Other solvents, known to people skilled in the art, may be used for a compound that provides the counterion to the α₂-adrenergic agonist, as appropriate. Solubilities of brimonidine free base, brimonidine pamoate, and brimonidine tartrate in water were measured, after 11 days on a twist shaker, to be 215.1 μg/ml, 20.7 μg/ml, and 41588 μg/ml, respectively. FIGS. 1, 2, and 3 show NMR spectra of brimonidine free base, pamoic acid, and brimonidine pamoate complex, respectively. FIG. 4 shows XRD spectra of pamoic acid (bottom curve), brimonidine free base (middle curve), and simple solid mixture of brimonidine and pamoic acid (top curve). FIG. 5 shows XRD spectra of pamoic acid (bottom curve), brimonidine free base (top curve), and complex of brimonidine and pamoic acid (middle curve).

### EXAMPLE 10

### Preparation of Various Complexes Comprising Brimonidine and Selected Counterions

In this experiment, various complexes comprising Brimonidine and counterions of one of the following acids were prepared: pamoic acid, capric acid, sebacic acid, hippuric acid, naproxen, 1-hydroxy-2-naphthoic acid, palmitic acid, and stearic acid. Variations of the procedure described in the following disclosure may be made within the skill of a person of ordinary skill in the art without departing from the scope of the present invention. Brimonidine free base in a preselected solvent was heated to about 60-70° C. The organic acid in another portion of the solvent was added into the heated mixture or was included in the original mixture before heating. The heating of the combined mixture was continued for an additional period, which was not critical. In certain embodiments, an antisolvent was added to the combined mixture, preferably at a lower temperature, to effect a precipitation of the complex of brimonidine and the counterion. It may be advantageous to remove a portion of the solvent and antisolvent to assist the precipitation. In certain other embodiments, the heated combined mixture was cooled down to a lower temperature, such as room temperature (or below) to effect the precipitation of the complex of brimonidine and the counterion. The precipitate was then filtered and dried to yield the final complex. The solubility of various complexes in water at the resulting pH is shown in Table 9.

TABLE 9
Solubility of Various Brimonidine Complexes

| Counterion | pH | Solubility (μg/mL) |
|---|---|---|
| None (brimonidine free | 8.1-8.9 | 48-200 |
| base) | | |
| pamoate | 7.2-7.5 | 2.2-38 |
| naphthoate | 5.3-5.4 | 411-413 |
| 1-hydroxy-2-naphtoate | 6.8 | 300 |
| stearate | 7.8-8.8 | 100-200 |
| palmitate | 7.1-8.8 | 6-173 |
| sebacate | 4.7-4.9 | 610-611 |

### EXAMPLE 11

### Preparation of Other Lots of Complexes Comprising Brimonidine and Selected Counterions

Several lots of complexes, in quantities of 1-4 grams, comprising brimonidine and selected counterions were prepared.

### EXAMPLE 11-1

### Complex Comprising Brimonidine and Pamoic Acid

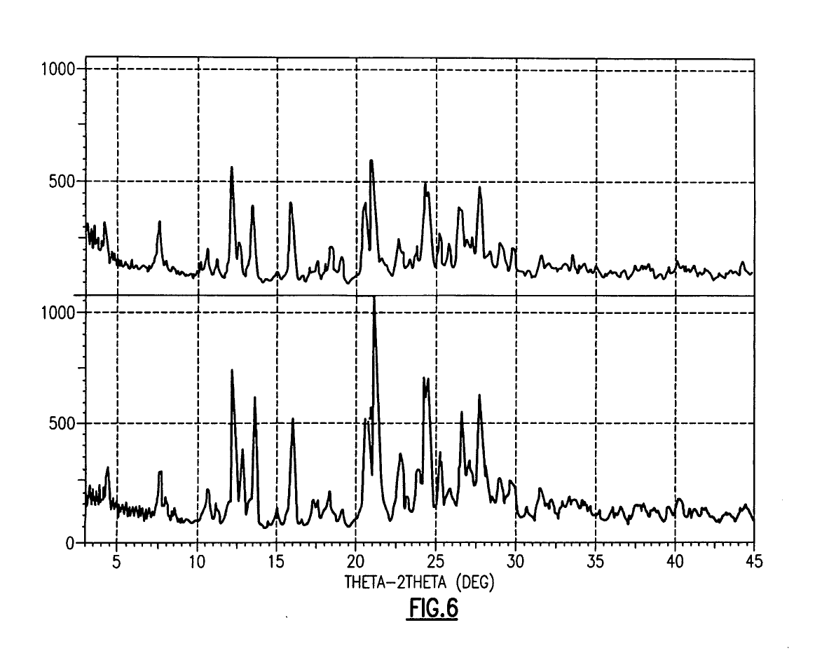

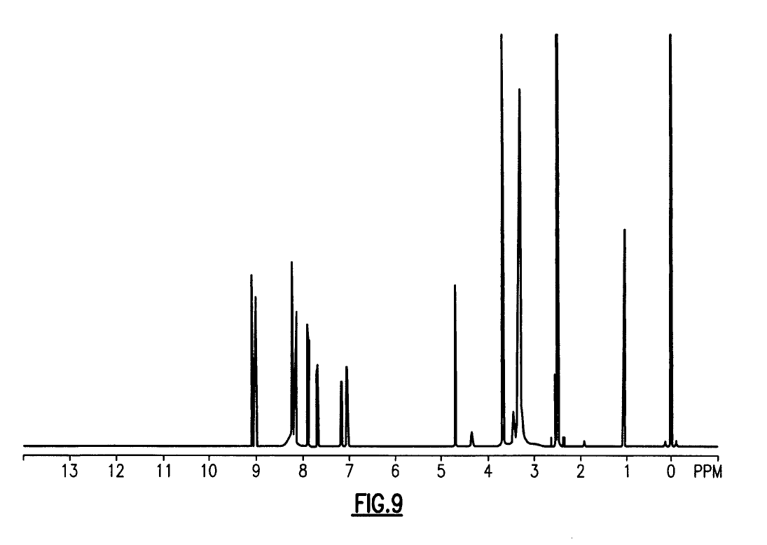

In a two-liter, three-neck round bottom flask equipped with overhead stirrer, heating mantle, condenser, temperature probe, and N₂ inlet, 2.0 g of brimonidine (lot BRMB-001 L08) was dissolved in ethanol (800 mL) at 65° C. Pamoic acid (1.05 eq, 7.5 mL, 0.5M in DMSO) was then added. The resulting solution was stirred for 10 minutes and then cooled at 20° C./h to ambient temperature. At 50° C., precipitation of solids was observed. The mixture stirred overnight at ambient temperature and was then filtered. The solids were then dried under vacuum at ambient temperature for 72 h affording 2.872 g (86% yield) of yellow solids (lot No. PDH-P-36(1)). XRD spectra of this material and another sample (lot JMS-A-23(1)) previously prepared are shown in FIG. 6. The spectra are consistent, indicating that the material was reproduced. A proton NMR spectrum of lot No. PDH-P-36(1) is shown in FIG. 9.

### EXAMPLE 11-2

### Complex Comprising Brimonidine and 1-Hydroxy-2-Naphthoic Acid

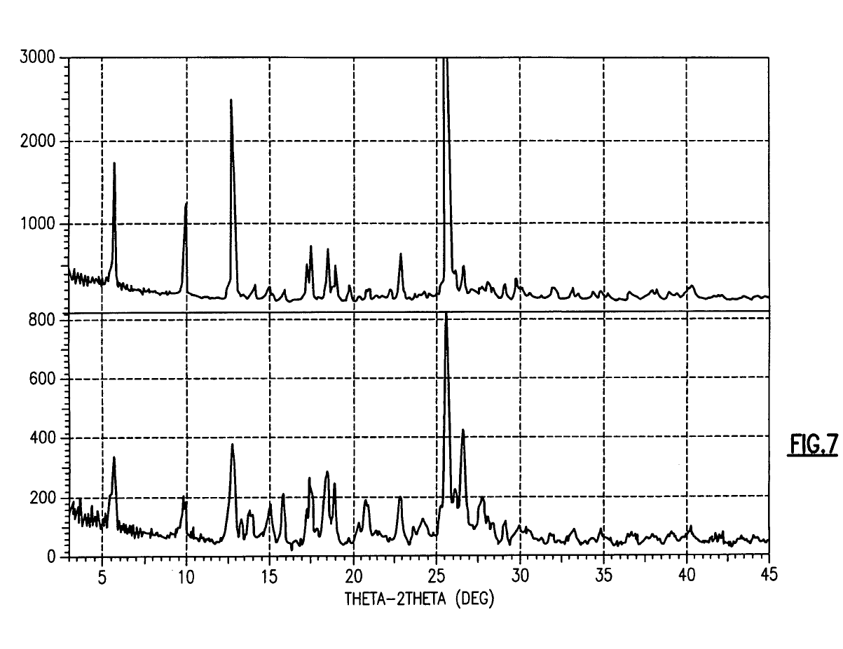

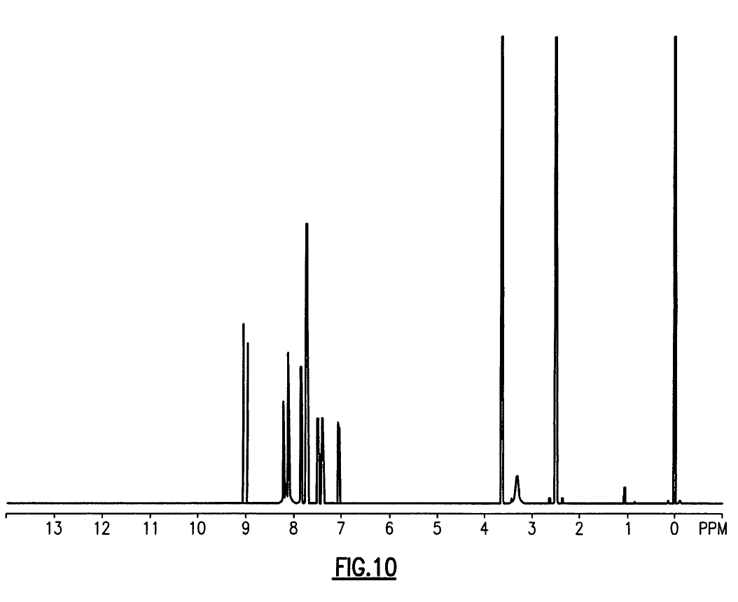

In a three-liter, three-neck round bottom flask equipped with overhead stirrer, heating mantle, condenser, temperature probe, and N₂ inlet, 1.25 g of brimonidine (lot BRMB-001 L08) was dissolved in ethanol (500 mL) at 65° C. 1-hydroxy-2-napthoic acid (1.05 eq, 17.9 mL, 0.25M in ethanol) was then added. The resulting solution was stirred for 10 minutes and then cooled to 55° C. Heptane (1 L) was then slowly added maintaining a reaction temperature of 50-55° C. No precipitation was observed. The solution was then seeded [lot PDH-P-37(1)] and cooled to ambient temperature at 20° C./h. At 35° C., precipitation began to thicken. After the reaction had reached ambient temperature, the mixture was further cooled to −5° C. using a MeCN/ice water bath for 2 h. The solids were then filtered and dried under vacuum at ambient temperature for 16 h affording 1.780 g (87% yield) of yellow solids (lot No. PDH-P-38(1)). XRD spectra of this material and another sample (lot JMS-A-22(1)) previously prepared are shown in FIG. 7. The spectra are consistent, indicating that the material was reproduced. A proton NMR spectrum of the sample of lot No. PDH-P-38(1) is shown in FIG. 10.

### EXAMPLE 11-3

### Complex Comprising Brimonidine and Diatrizoic Acid

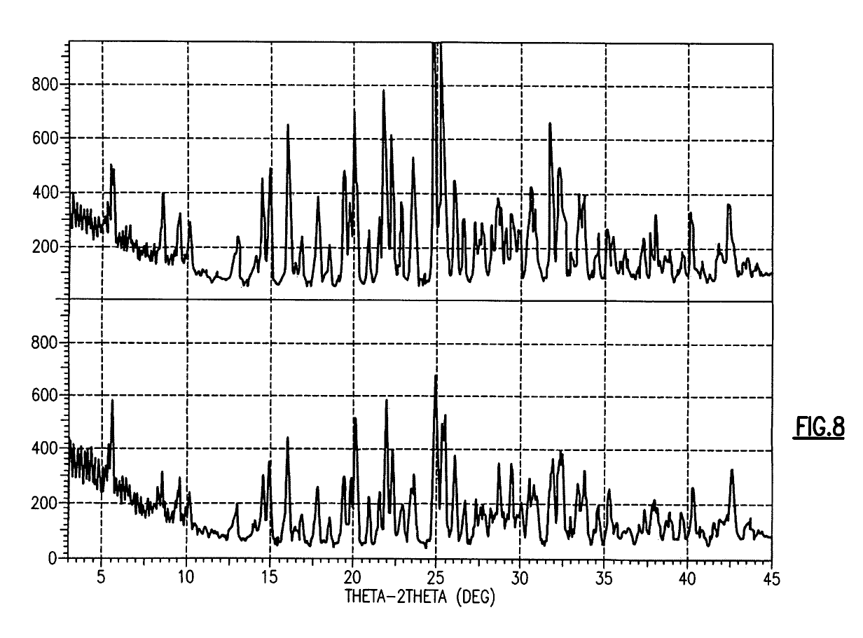

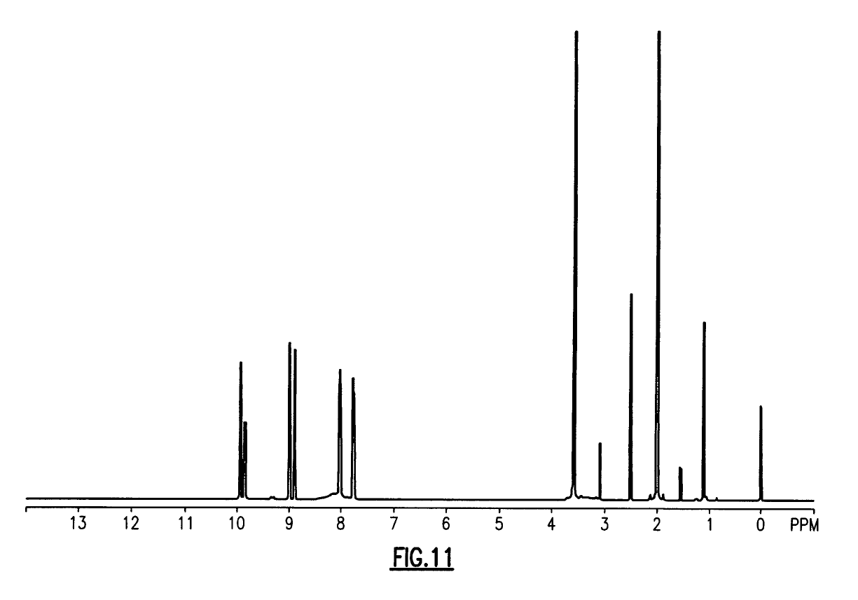

In a two-liter, three-neck round bottom flask equipped with overhead stirrer, heating mantle, condenser, temperature probe, and N₂ inlet, 1.25 g of brimonidine (lot BRMB-001 L08) was dissolved in methanol (350 mL) at 65° C. The solution was then cooled to 60° C. Diatrizoic acid (1.05 eq, 113 mL, 0.04M in methanol) was then added. The resulting solution was stirred for 10 minutes and then cooled to 50° C. MTBE (700 mL) was then slowly added maintaining a reaction temperature of 48-50° C. The reaction was slightly cloudy upon completion of the addition. The reaction was then seeded (lot HAL-B-100(7A)). Precipitation initiated shortly after seeding at 50° C. The mixture was then cooled to ambient temperature at 20° C./h and stirred overnight. The mixture was then cooled to 5° C. for 1 h using an ice water bath. The solids were then filtered and dried under vacuum at ambient temperature for 20 h affording 3.170 g (81% yield) of yellow solids (lot No. PDH-P-39(1)). XRD spectra of this material and another sample (lot JMS-A-63(1)) previously prepared are shown in FIG. 8. The spectra are consistent, indicating that the material was reproduced. A proton NMR spectrum of lot PDH-P-39(1) is shown in FIG. 11.

While specific embodiments of the present invention have been described in the foregoing, it will be appreciated by those skilled in the art that many equivalents, modifications, substitutions, and variations may be made thereto without departing from the spirit and scope of the invention as defined in the appended claims.

What is claimed is:

1. A complex comprising at least an α₂-adrenergic receptor agonist and a compound that provides an opposite charge to a charge on the α₂-adrenergic receptor agonist, wherein the complex is charge neutral as a whole and has a solubility in a range from about 0.3 μg/ml to about 2.5 mg/ml in water at pH of about 7 and temperature of about 25° C.

2. The complex of claim 1, wherein said compound is selected from the group consisting of carboxylic acids other than fatty acids, sulfonic acids, phosphonic acids, and combinations thereof.

3. The complex of claim 1, wherein said at least an α₂-adrenergic receptor agonist is selected from the group consisting of quinoxalines, imino-imidazolines, imidazolines, imidazoles, azepines, thiazines, oxazolines, guanidines, catecholamines, derivatives thereof, combinations thereof, and mixtures thereof.

4. The complex of claim 1, wherein said at least an α₂-adrenergic receptor agonist comprises a quinoxaline or a derivative thereof.

5. The complex of claim 4, wherein said at least an α₂-adrenergic receptor agonist comprises a material having Formula I 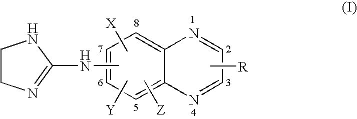
wherein the 2-imidazolin-2-ylamino group is attached to the 5-, 6-, 7-, or 8-position of the quinoxaline nucleus; X, Y, and Z are attached to the remaining 5-, 6-, 7-, and 8-positions; each of X, Y, and Z is independently selected from the group consisting of hydrogen, halogen, lower alkyl, lower alkoxy, and trifluoromethyl; and R comprises a substituent attached to the 2- or 3-position of the quinoxaline nucleus and is selected from the group consisting of hydrogen, lower alkyl, and lower alkoxy.

6. The complex of claim 5, wherein the halogen comprises bromine.

7. The complex of claim 5, wherein each of the lower alkyl and lower alkoxy groups comprises one to five carbon atoms.

8. The complex of claim 4, wherein said at least an α₂-adrenergic receptor agonist comprises a material having Formula II

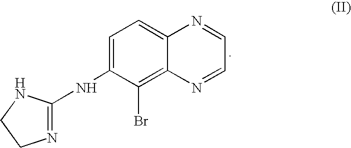

9. The complex of claim 1, wherein said compound is selected from the group consisting of pamoic acid, sebacic acid, hippuric acid, capric acid, mandelic acid, (S)-(+)-2-(6-methoxy-2-naphthyl)propionic acid (naproxen), dichloroacetic acid, adipic acid, 4-acetamidobenzoic acid, cinnamic acid, dodecylsulfuric acid, salicylic acid, gentisic acid, naphthalene-2-sulfonic acid, 1-hydroxy-2-naphthoic acid, docosahexaenoic acid (“DHA”), arachidonic acid, eicosenoic, cholesteric acid, taurocholic acid, taurodeoxycholic acid, taurochenodeoxycholic acid, glycocholic acid, glycochenodeoxycholic acid, diatrizoic acid (iodamide), iobenzamic acid, iocarmic acid, iocetamic acid, iodipamide (3,3′-(adipoyldiimino)bis(2,4,6-triiododenzoic acid)), iodoalphionic acid, iodobenzoic acid, ioglycamic acid, iomeglamic acid, iopanoic acid, iophenoxic acid, iopronic acid, iothalamic acid, ioxaglic acid, ipodate (β-(3-dimethylaminomethyleneamino-2,4,6-triiodophenyl)propionic acid, ethylenediaminetetraacetic acid (“EDTA”), diethylenetriaminepentaacetic acid (“DTPA”), 1,4,7,10-tetraazacyclododecane-N,N′,N″,N′″-tetraacetic acid (“DOTA”), benzyloxypropionictetraacetic acid (“BOPTA”), triethylenetetraminehexaacetic acid (“TTHA”), 1,2-diaminocyclohexane-N,N,N′,N′-tetraacetic acid, N-(2-hydroxyethyl)ethylenediaminetriacetic acid, nitrilotriacetic acid, ethylene-bis(oxyethylenenitrilo)tetraacetic acid (“EGTA”), 1,4,7,10-tetraazacyclododecane-N,N′,N″-triacetic acid (“DO3A”), 1,4,7-tris(carboxymethyl)-10-(2′-hydroxy)propyl)-1,4,7,10-tetraazocyclodecane (“HP-DO3A”), 1,4,7-triazacyclononane-N,N′,N-triacetic acid (“NOTA”), 1,4,8,1-tetraazacyclotetradecane-N,N′,N″,N′″-tetraacetic acid (“TETA”), combinations thereof, and mixtures thereof.

10. The complex of claim 3, wherein said compound is selected from the group consisting of pamoic acid, sebacic acid, hippuric acid, capric acid, mandelic acid, (S)-(+)-2-(6-methoxy-2-naphthyl)propionic acid (naproxen), dichloroacetic acid, adipic acid, 4-acetamidobenzoic acid, cinnamic acid, dodecylsulfuric acid, salicylic acid, gentisic acid, naphthalene-2-sulfonic acid, 1-hydroxy-2-naphthoic acid, docosahexaenoic acid (“DHA”), arachidonic acid, eicosenoic acid, cholesteric acid, taurocholic acid, taurodeoxycholic acid, taurochenodeoxycholic acid, glycocholic acid, glycochenodeoxycholic acid, diatrizoic acid (iodamide), iobenzamic acid, iocarmic acid, iocetamic acid, iodipamide (3,3′-(adipoyldiimino)bis(2,4,6-triiododenzoic acid)), iodoalphionic acid, iodobenzoic acid, ioglycamic acid, iomeglamic acid, iopanoic acid, iophenoxic acid, iopronic acid, iothalamic acid, ioxaglic acid, ipodate (β-(3-dimethylaminomethyleneamino-2,4,6-triiodophenyl)propionic acid, ethylenediaminetetraacetic acid (“EDTA”), diethylenetriaminepentaacetic acid (“DTPA”), 1,4,7,10-tetraazacyclododecane-N,N′,N″,N′″-tetraacetic acid (“DOTA”), benzyloxypropionictetraacetic acid (“BOPTA”), triethylenetetraminehexaacetic acid (“TTHA”), 1,2-diaminocyclohexane-N,N,N′,N′-tetraacetic acid, N-(2-hydroxyethyl)ethylenediaminetriacetic acid, nitrilotriacetic acid, ethylene-bis(oxyethylenenitrilo)tetraacetic acid (“EGTA”), 1,4,7,10-tetraazacyclododecane-N,N′,N″-triacetic acid (“DO3A”), 1,4,7-tris(carboxymethyl)-10-(2′-hydroxy)propyl)-1,4,7,10-tetraazocyclodecane (“HP-DO3A”), 1,4,7-triazacyclononane-N,N′,N-triacetic acid (“NOTA”), 1,4,8,11-tetraazacyclotetradecane-N,N′,N″,N′″-tetraacetic acid (“TETA”), combinations thereof, and mixtures thereof.

11. The complex of claim 3, wherein said compound is selected from the group consisting of pamoic acid, sebacic acid, hippuric acid, mandelic acid, (S)-(+)-2-(6-methoxy-2-naphthyl)propionic acid (naproxen), dichloroacetic acid, adipic acid, 4-acetamidobenzoic acid, cinnamic acid, dodecylsulfuric acid, salicylic acid, gentisic acid, naphthalene-2-sulfonic acid, 1-hydroxy-2-naphthoic acid, combinations thereof, and mixture thereof.

12. The complex of claim 3, wherein said compound comprises pamoic acid.

13. The complex of claim 3, wherein said compound is selected from the group consisting of cholesteric acid, taurocholic acid, taurodeoxycholic acid, taurochenodeoxycholic acid, glycocholic acid, glycochenodeoxycholic acid, combinations thereof, and mixtures thereof.

14. The complex of claim 3, wherein said compound is selected from the group consisting of diatrizoic acid (iodamide), iobenzamic acid, iocarmic acid, iocetamic acid, iodipamide (3,3′-(adipoyldiimino)bis(2,4,6-triiododenzoic acid)), iodoalphionic acid, iodobenzoic acid, ioglycamic acid, iomeglamic acid, iopanoic acid, iophenoxic acid, iopronic acid, iothalamic acid, ioxaglic acid, ipodate (β-(3-dimethylaminomethyleneamino-2,4,6-triiodophenyl)propionic acid, ethylenediaminetetraacetic acid (“EDTA”), diethylenetriaminepentaacetic acid (“DTPA”), 1,4,7,10-tetraazacyclododecane-N,N′,N″,N′″-tetraacetic acid (“DOTA”), benzyloxypropionictetraacetic acid (“BOPTA”), triethylenetetraminehexaacetic acid (“TTHA”), 1,2-diaminocyclohexane-N,N,N′,N′-tetraacetic acid, N-(2-hydroxyethyl)ethylenediaminetriacetic acid, nitrilotriacetic acid, ethylene-bis(oxyethylenenitrilo)tetraacetic acid (“EGTA”), 1,4,7,10-tetraazacyclododecane-N,N′,N″-triacetic acid (“DO3A”), 1,4,7-tris(carboxymethyl)-10-(2′-hydroxy)propyl)-1,4,7,10-tetraazocyclodecane (“HP-DO3A”), 1,4,7-triazacyclononane-N,N′,N-triacetic acid (“NOTA”), 1,4,8,11-tetraazacyclotetradecane-N,N′,N″,N′″-tetraacetic acid (“TETA”), combinations thereof, and mixtures thereof.

15. The complex of claim 5, wherein said compound is selected from the group consisting of pamoic acid, sebacic acid, hippuric acid, capric acid, mandelic acid, (S)-(+)-2-(6-methoxy-2-naphthyl)propionic acid (naproxen), dichloroacetic acid, adipic acid, 4-acetamidobenzoic acid, cinnamic acid, dodecylsulfuric acid, salicylic acid, gentisic acid, naphthalene-2-sulfonic acid, 1-hydroxy-2-naphthoic acid, docosahexaenoic acid (“DHA”), arachidonic acid, eicosenoic acid, cholesteric acid, taurocholic acid, taurodeoxycholic acid, taurochenodeoxycholic acid, glycocholic acid, glycochenodeoxycholic acid, diatrizoic acid (iodamide), iobenzamic acid, iocarmic acid, iocetamic acid, iodipamide (3,3′-(adipoyldiimino)bis(2,4,6-triiododenzoic acid)), iodoalphionic acid, iodobenzoic acid, ioglycamic acid, iomeglamic acid, iopanoic acid, iophenoxic acid, iopronic acid, iothalamic acid, ioxaglic acid, ipodate (β-(3-dimethylaminomethyleneamino-2,4,6-triiodophenyl)propionic acid, ethylenediaminetetraacetic acid (“EDTA”), diethylenetriaminepentaacetic acid (“DTPA”), 1,4,7,10-tetraazacyclododecane-N,N′,N″,N′″-tetraacetic acid (“DOTA”), benzyloxypropionictetraacetic acid (“BOPTA”), triethylenetetraminehexaacetic acid (“TTHA”), 1,2-diaminocyclohexane-N,N,N′,N′-tetraacetic acid, N-(2-hydroxyethyl)ethylenediaminetriacetic acid, nitrilotriacetic acid, ethylene-bis(oxyethylenenitrilo)tetraacetic acid (“EGTA”), 1,4,7,10-tetraazacyclododecane-N,N′,N″-triacetic acid (“DO3A”), 1,4,7-tris(carboxymethyl)-10-(2′-hydroxy)propyl)-1,4,7,10-tetraazocyclodecane (“HP-DO3A”), 1,4,7-triazacyclononane-N,N′,N-triacetic acid (“NOTA”), 1,4,8,11-tetraazacyclotetradecane-N,N′,N″,N′″-tetraacetic acid (“TETA”), combinations thereof, and mixtures thereof.

16. The complex of claim 8, wherein said compound is selected from the group consisting of pamoic acid, sebacic acid, hippuric acid, capric acid, mandelic acid, (S)-(+)-2-(6-methoxy-2-naphthyl)propionic acid (naproxen), dichloroacetic acid, adipic acid, 4-acetamidobenzoic acid, cinnamic acid, dodecylsulfuric acid, salicylic acid, gentisic acid, naphthalene-2-sulfonic acid, 1-hydroxy-2-naphthoic acid, docosahexaenoic acid (“DHA”), arachidonic acid, eicosenoic acid, cholesteric acid, taurocholic acid, taurodeoxycholic acid, taurochenodeoxycholic acid, glycocholic acid, glycochenodeoxycholic acid, diatrizoic acid (iodamide), iobenzamic acid, iocarmic acid, iocetamic acid, iodipamide (3,3′-(adipoyidiimino)bis(2,4,6-triiododenzoic acid)), iodoalphionic acid, iodobenzoic acid, ioglycamic acid, iomeglamic acid, iopanoic acid, iophenoxic acid, iopronic acid, iothalamic acid, ioxaglic acid, ipodate (β-(3-dimethylaminomethyleneamino-2,4,6-triiodophenyl)propionic acid, ethylenediaminetetraacetic acid (“EDTA”), diethylenetriaminepentaacetic acid (“DTPA”), 1,4,7,10-tetraazacyclododecane-N,N′,N″,N′″-tetraacetic acid (“DOTA”), benzyloxypropionictetraacetic acid (“BOPTA”), triethylenetetraminehexaacetic acid (“TTHA”), 1,2-diaminocyclohexane-N,N,N′,N′-tetraacetic acid, N-(2-hydroxyethyl)ethylenediaminetriacetic acid, nitrilotriacetic acid, ethylene-bis(oxyethylenenitrilo)tetraacetic acid (“EGTA”), 1,4,7,10-tetraazacyclododecane-N,N′,N″-triacetic acid (“DO3A”), 1,4,7-tris(carboxymethyl)-10-(2′-hydroxy)propyl)-1,4,7,10-tetraazocyclodecane (“HP-DO3A”), 1,4,7-triazacyclononane-N,N′,N-triacetic acid (“NOTA”), 1,4,8,11-tetraazacyclotetradecane-N,N′,N″,N′″-tetraacetic acid (“TETA”), combinations thereof, and mixtures thereof.

17. The complex of claim 8, wherein said compound comprises pamoic acid.

18. The complex of claim 1, wherein the complex has a solubility in a range from about 1 μg/ml to about 1.5 mg/ml in water at pH of about 7 and temperature of about 25° C.

19. The complex of claim 1, wherein the complex has a solubility in a range from about 1 μg/ml to about 1 mg/ml in water at pH of about 7 and temperature of about 25° C.

20. A composition comprising a pharmaceutically acceptable carrier and a complex that comprises at least an α₂-adrenergic receptor agonist and a compound that provides an opposite charge to a charge on the α₂-adrenergic receptor agonist, wherein the complex is charge neutral as a whole and has a solubility in a range from about 0.3 μg/ml to about 2.5 mg/ml in water at pH of about 7 and temperature of about 25° C.

21. The composition of claim 20, wherein said compound is selected from the group consisting of carboxylic acids other than fatty acids, sulfonic acids, phosphonic acids, and combinations thereof.

22. The composition of claim 20, wherein said at least an α₂-adrenergic receptor agonist is selected from the group consisting of quinoxalines, imino-imidazolines, imidazolines, imidazoles, azepines, thiazines, oxazolines, guanidines, catecholamines, derivatives thereof, combinations thereof, and mixtures thereof.

23. The composition of claim 20, wherein said at least an α₂-adrenergic receptor agonist comprises a quinoxaline or a derivative thereof.

24. The composition of claim 20, wherein said at least an α₂-adrenergic receptor agonist comprises a material having Formula I 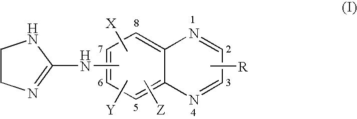
wherein the 2-imidazolin-2-ylamino group is attached to the 5-, 6-, 7-, or 8-position of the quinoxaline nucleus; X, Y, and Z are attached to the remaining 5-, 6-, 7-, and 8-positions; each of X, Y, and Z is independently selected from the group consisting of hydrogen, halogen, lower alkyl, lower alkoxy, and trifluoromethyl; and R comprises a substituent attached to the 2- or 3-position of the quinoxaline nucleus and is selected from the group consisting of hydrogen, lower alkyl, and lower alkoxy.

25. The composition of claim 20, wherein said at least an α₂-adrenergic receptor agonist comprises a material having Formula II

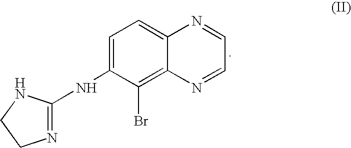

26. The composition of claim 25, wherein said compound is selected from the group consisting of pamoic acid, sebacic acid, hippuric acid, capric acid, mandelic acid, (S)-(+)-2-(6-methoxy-2-naphthyl)propionic acid (naproxen), dichloroacetic acid, adipic acid, 4-acetamidobenzoic acid, cinnamic acid, dodecylsulfuric acid, salicylic acid, gentisic acid, naphthalene-2-sulfonic acid, 1-hydroxy-2-naphthoic acid, docosahexaenoic acid (“DHA”), arachidonic acid, eicosenoic acid, cholesteric acid, taurocholic acid, taurodeoxycholic acid, taurochenodeoxycholic acid, glycocholic acid, glycochenodeoxycholic acid, diatrizoic acid (iodamide), iobenzamic acid, iocarmic acid, iocetamic acid, iodipamide (3,3′-(adipoyldiimino)bis(2,4,6-triiododenzoic acid)), iodoalphionic acid, iodobenzoic acid, ioglycamic acid, iomeglamic acid, iopanoic acid, iophenoxic acid, iopronic acid, iothalamic acid, ioxaglic acid, ipodate (β-(3-dimethylaminomethyleneamino-2,4,6-triiodophenyl)propionic acid, ethylenediaminetetraacetic acid (“EDTA”), diethylenetriaminepentaacetic acid (“DTPA”), 1,4,7,10-tetraazacyclododecane-N,N′,N″,N′″-tetraacetic acid (“DOTA”), benzyloxypropionictetraacetic acid (“BOPTA”), triethylenetetraminehexaacetic acid (“TTHA”), 1,2-diaminocyclohexane-N,N,N′,N′-tetraacetic acid, N-(2-hydroxyethyl)ethylenediaminetriacetic acid, nitrilotriacetic acid, ethylene-bis(oxyethylenenitrilo)tetraacetic acid (“EGTA”), 1,4,7,10-tetraazacyclododecane-N,N′,N″-triacetic acid (“DO3A”), 1,4,7-tris(carboxymethyl)-10-(2′-hydroxy)propyl)-1,4,7,10-tetraazocyclodecane (“HP-DO3A”), 1,4,7-triazacyclononane-N,N′,N-triacetic acid (“NOTA”), 1,4,8,11-tetraazacyclotetradecane-N,N′,N″,N′″-tetraacetic acid (“TETA”), combinations thereof, and mixtures thereof.

27. The composition of claim 22, wherein said compound is selected from the group consisting of pamoic acid, sebacic acid, hippuric acid, mandelic acid, (S)-(+)-2-(6-methoxy-2-naphthyl)propionic acid (naproxen), dichloroacetic acid, adipic acid, 4-acetamidobenzoic acid, cinnamic acid, dodecylsulfuric acid, salicylic acid, gentisic acid, naphthalene-2-sulfonic acid, 1-hydroxy-2-naphthoic acid, combinations thereof, and mixture thereof.

28. The composition of claim 22, wherein said compound is selected from the group consisting of cholesteric acid, taurocholic acid, taurodeoxycholic acid, taurochenodeoxycholic acid, glycocholic acid, glycochenodeoxycholic acid, combinations thereof, and mixtures thereof.

29. The composition of claim 22, wherein said compound is selected from the group consisting of diatrizoic acid (iodamide), iobenzamic acid, iocarmic acid, iocetamic acid, iodipamide (3,3′-(adipoyidiimino)bis(2,4,6-triiododenzoic acid)), iodoalphionic acid, iodobenzoic acid, ioglycamic acid, iomeglamic acid, iopanoic acid, iophenoxic acid, iopronic acid, iothalamic acid, ioxaglic acid, ipodate (β-(3-dimethylaminomethyleneamino-2,4,6-triiodophenyl)propionic acid, ethylenediaminetetraacetic acid (“EDTA”), diethylenetriaminepentaacetic acid (“DTPA”), 1,4,7,10-tetraazacyclododecane-N,N′,N″,N′″-tetraacetic acid (“DOTA”), benzyloxypropionictetraacetic acid (“BOPTA”), triethylenetetraminehexaacetic acid (“TTHA”), 1,2-diaminocyclohexane-N,N,N′,N′-tetraacetic acid, N-(2-hydroxyethyl)ethylenediaminetriacetic acid, nitrilotriacetic acid, ethylene-bis(oxyethylenenitrilo)tetraacetic acid (“EGTA”), 1,4,7,10-tetraazacyclododecane-N,N′,N″-triacetic acid (“DO3A”), 1,4,7-tris(carboxymethyl)-10-(2′-hydroxy)propyl)-1,4,7,10-tetraazocyclodecane (“HP-DO3A”), 1,4,7-triazacyclononane-N,N′,N-triacetic acid (“NOTA”), 1,4,8,11-tetraazacyclotetradecane-N,N′,N″,N′″-tetraacetic acid (“TETA”), combinations thereof, and mixtures thereof.

30. The composition of claim 20, wherein the complex has a solubility in a range from about 1 μg/ml to about 1.5 mg/ml in water at pH of about 7 and temperature of about 25° C.

31. The composition of claim 30, wherein a portion of said complex remains in a solid phase for a period longer than one day after said complex has been in contact with said pharmaceutically acceptable carrier.

32. A method for providing neuroprotection to a neurological tissue, said method comprising administering into a subject in need of such neuroprotection a composition of claim 20.

33. The method of claim 32, wherein said method comprising administering into a subject in need of such neuroprotection a composition of claim 22.

34. The method of claim 32, wherein said method comprising administering into a subject in need of such neuroprotection a composition of claim 24.

35. The method of claim 32, wherein said method comprising administering into a subject in need of such neuroprotection a composition of claim 25.

36. The method of claim 32, wherein said method comprising administering into a subject in need of such neuroprotection a composition of claim 26.

37. The method of claim 32, wherein said method comprising administering into a subject in need of such neuroprotection a composition of claim 31.

38. The method of claim 32, wherein said neuroprotection prevents progressive damage to cells or components of the optic nerve resulting from a back-of-the-eye pathological condition.

39. The method of claim 38, wherein said damage results from glaucoma, retinitis pigmentosa, AMD, diabetic retinopathy, diabetic macular edema, and combinations thereof.

40. The method of claim 35, wherein said neuroprotection prevents progressive damage to cells or components of the optic nerve resulting from a back-of-the-eye pathological condition.

41. The method of claim 40, wherein said damage results from glaucoma, retinitis pigmentosa, AMD, diabetic retinopathy, diabetic macular edema, and combinations thereof.

42. A method for producing a composition comprising a complex that comprises at least an α₂-adrenergic receptor agonist and a counterion, the method comprising:
(a) providing said complex that has a solubility in a medium and a portion of which complex remains in a solid phase for a period longer than one day after said complex has been in contact with said medium; and
(b) dispersing an amount of said complex in a sufficient amount of said medium to produce said composition to achieve a predetermined concentration of said complex in said medium.

43. The method of claim 42, wherein said at least an α₂-adrenergic receptor agonist comprises a material having Formula I.

44. The method of claim 42, wherein said at least an α₂-adrenergic receptor agonist comprises a material having Formula II.

45. A method for producing a composition comprising a complex that comprises at least an α₂-adrenergic receptor agonist and a counterion, the method comprising:
(a) providing said at least an α₂-adrenergic receptor agonist and a compound that is ionizable to said counterion;
(b) mixing said at least an α₂-adrenergic receptor agonist and said compound to produce the complex;
(c) adjusting a pH of a mixture of said at least an α₂-adrenergic receptor agonist and said compound to a pH in a range from about 7 to about 7.5;
(d) recovering a precipitate of the complex that comprises said at least an α₂-adrenergic receptor agonist and said counterion; and
(e) dispersing said precipitate in an amount of a medium to produce said composition to achieve a predetermined concentration of said complex in said medium, wherein a portion of said complex remains in a solid phase for a period longer than one day after said complex has been in contact with said medium.

46. The method of claim 45, wherein said at least an α₂-adrenergic receptor agonist comprises a material having Formula I.

47. The method of claim 45, wherein said at least an α₂-adrenergic receptor agonist comprises a material having Formula II.
